(19) United States
(12) Patent Application Publication
(10) Pub. No.: US 2024/0398573 A1
(43) Pub. Date: Dec. 5, 2024

(54) JOINT REPLACEMENT SYSTEM ENABLING JOINT MOVEMENT

(71) Applicant: John G. Rupolo, Floral Park, NY (US)

(72) Inventor: John G. Rupolo, Floral Park, NY (US)

(21) Appl. No.: 18/205,649

(22) Filed: Jun. 5, 2023

(51) Int. Cl. A61F 2/30; A61F 2/38; A61L 27/16

(52) U.S. Cl. CPC A61F 2/30734; A61F 2/38; A61L 27/165; A61F 2002/30548; A61F 2002/30581

(57) ABSTRACT

A joint replacement system includes an insert configured to be disposed on a surface of an articular limb joint, and a condyle configured to contain a biocompatible fluid and to be disposed on an opposing surface of a limb joint, wherein the condyle and the insert are aligned to enable articulation of the limb joint.

### TECHNICAL FIELD

The present disclosure relates generally to the field of joint replacement systems. More specifically, an aspect of the present disclosure provides a joint replacement system for a knee configured to enable the efficient movement of the knee joint.

### BACKGROUND

Efficient joint movement and mobility aid in lowering physical injury in that in the event of an elevated range of joint movement, joint strength is greatly reduced, thus increasing the risk of injury. While a majority of lower lumbar pain may be attributed to muscle related issues, a large portion of patients who experience lower lumbar pain suffer from various joint problems that, in turn, lead to compromised lower lumbar segments. Certain joints, such as the knee, play a substantial role in determining how an individual's back functions and feels. Over time, wear and tear of the joints associated with age and movement may lead to the development of substantial and, in some cases, chronic lower lumbar pain that can make engaging in day-to-day activities painful and burdensome.

Accordingly, there is interest in joint replacement systems that mitigate the degradation of joints over time, in turn reducing the likelihood and severity of lower lumbar pain and promoting longevity of healthy joint movement.

### SUMMARY

An aspect of the present disclosure provides a joint replacement system. The joint replacement system includes an insert configured to be disposed on an articular surface of a limb joint, and a condyle configured to contain a biocompatible fluid and to be disposed on an opposing surface of the limb joint. The condyle and the insert are aligned to enable articulation of the limb joint.

In accordance with aspects of the disclosure, the insert may be secured on the articular surface of the limb joint by a biocompatible adhesive.

In an aspect of the present disclosure, the insert may be secured on the articular surface of the limb joint by a biocompatible adhesive film.

In another aspect of the present disclosure, the insert may be made at least in part from polytetrafluoroethylene.

In yet another aspect of the present disclosure, the limb joint may be a knee joint.

In a further aspect of the present disclosure, the condyle may be configured to selectively receive an amount of the biocompatible fluid therein.

In yet a further aspect of the present disclosure, the condyle may include a port disposed along an exterior surface of the condyle.

In accordance with aspects of the disclosure, the biocompatible fluid may be a synthetic synovial fluid.

In an aspect of the present disclosure, the condyle may be made at least in part from a rubberized biocompatible material.

In another aspect of the present disclosure, the insert and the condyle may be configured to articulate against one another.

In yet another aspect of the present disclosure, the insert and the condyle may be configured to be in a contact relationship with one another when the limb joint is in both a flexed and extended position.

In another aspect, the present disclosure provides a condyle configured to be disposed on an opposing surface of a limb joint. The condyle includes an exterior surface, an interior defining a volume for containing a biocompatible fluid, and an injection port disposed along the exterior surface of the condyle.

In another aspect of the present disclosure, the exterior surface of the condyle may be made at least in part from a rubberized biocompatible material.

In a further aspect of the present disclosure, the biocompatible fluid may be a synthetic synovial fluid.

In yet another aspect, the present disclosure provides a method for improving movement of a limb joint of a patent. The method includes attaching a first surface of an insert to an articular surface of the limb joint and attaching a first surface of a condyle to an opposing surface of the limb joint, the condyle containing a fluid therein. A second surface of the insert is in a contact relationship with a second surface of the condyle when the limb joint is in both a flexed and extended position.

In another aspect of the present disclosure, attaching the first surface of the insert to the articular surface may include applying a biocompatible adhesive between the first surface of the insert and the articular surface.

In a further aspect of the present disclosure, attaching the first surface of the condyle to the opposing surface may include applying a biocompatible adhesive between the first surface of the condyle and the opposing surface.

In yet another aspect of the present disclosure, injecting fluid into the condyle may occur through a port on an exterior surface of the condyle.

### BRIEF DESCRIPTION OF THE DRAWINGS

A better understanding of the features and advantages of the present disclosure will be obtained by reference to the following detailed description that sets forth illustrative aspects, in which the principles of the present disclosure are utilized, and the accompanying drawings of which:

FIG. 1 is a diagram of a medial to lateral flexion view of a joint replacement system, in accordance with aspects of the disclosure;

FIG. 2 is a diagram of a lateral to medial non-flexion view of the joint replacement system of FIG. 1;

FIG. 3 is a diagram of a posterior non-flexion view of the joint replacement system of FIG. 1; and

FIG. 4 is a diagram of an anterior flexion view of a condyle filled with a biocompatible fluid positioned on an articular surface of a limb joint of FIG. 1.

### DETAILED DESCRIPTION

The present disclosure relates generally to the field of joint replacement systems. More specifically, an aspect of the present disclosure provides a joint replacement system that enables the efficient movement of a limb joint, such as a knee. The disclosed joint replacement system mitigates the degradation of joints over time, in turn minimizing the likelihood and severity of lower lumbar pain and promoting longevity of healthy joint movement. The disclosed joint replacement system weighs less than metal reinforcements of the joint, providing the benefit, over metal reinforcement, of creating a symmetric weight distribution and not causing a transfer of weight to the opposite side. This provides the additional benefit of minimizing stress and improving the range of motion. Unlike metal reinforcement of the joint, the disclosed joint replacement system will not transfer weight to the opposite limb and will not cause issues with the lower back.

Aspects of the present disclosure are described in detail with reference to the drawings wherein like reference numerals identify similar or identical elements.

Although the present disclosure will be described in terms of specific aspects and examples, it will be readily apparent to those skilled in this art that various modifications, rearrangements, and substitutions may be made without departing from the spirit of the present disclosure.

For purposes of promoting an understanding of the principles of the present disclosure, reference will now be made to exemplary aspects illustrated in the drawings, and specific language will be used to describe the same. It will nevertheless be understood that no limitation of the scope of the present disclosure is thereby intended. Any alterations and further modifications of the features illustrated herein, and any additional applications of the principles of the present disclosure as illustrated herein, which would occur to one skilled in the relevant art and having possession of this disclosure, are to be considered within the scope of the present disclosure.

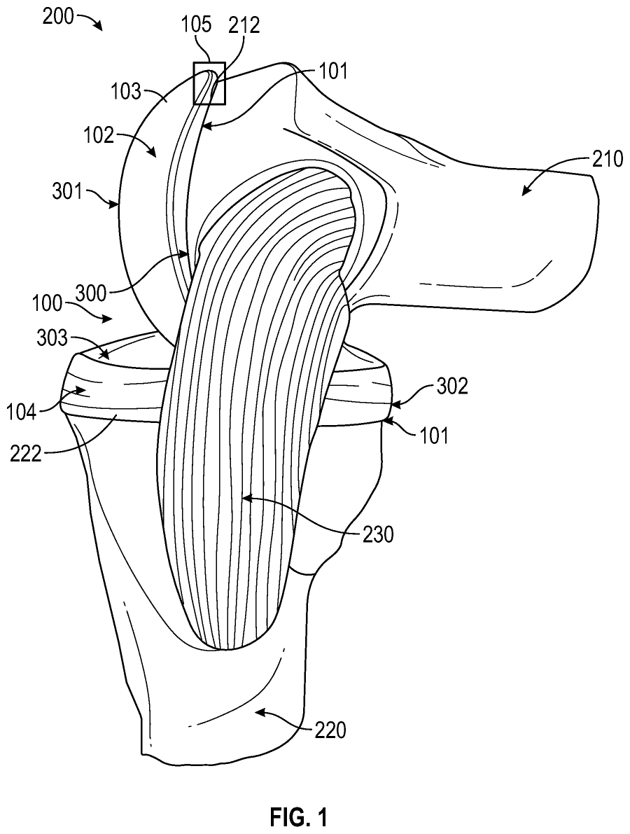

Referring to FIG. 1, a diagram of a medial to lateral flexion view of a joint replacement system 100 is shown. The joint replacement system 100 generally includes an insert 104 configured to be disposed on an articular surface 222 of a limb joint 200 (e.g., a knee) and a condyle 102 configured to contain a biocompatible fluid 103. Insert 104 includes a first surface 303 and a second surface 302. First surface 303 is configured to contact condyle 102 when the system 100 is installed in the limb joint 200, and second surface 302 is configured to be disposed on the articular surface 222 of limb joint 200. Condyle 102 includes a first surface 301 and a second surface 300. Second surface 300 of condyle 102 is configured to be disposed on an opposing surface 212 of limb joint 200, and first surface 301 of condyle 102 is configured to contact first surface 303 of insert 104 when the system 100 is installed in limb joint 200 such that insert 104 and condyle 102 are aligned to enable articulation of limb joint 200.

Insert 104 serves as an artificial replacement of a patient's articular cartilage which may be damaged by physical trauma or disease, or which may simply be worn out. Insert 104 may be formed to match the shape of the natural articular cartilage that it serves to replace and may be made from any lubricative biocompatible material having sufficient strength to withstand the stresses present in limb joint 200. Polytetrafluoroethylene, sometimes marketed under the trademark Teflon™, is an example of a resilient material that possesses a small likelihood of deformity and offers effective lubricative qualities.

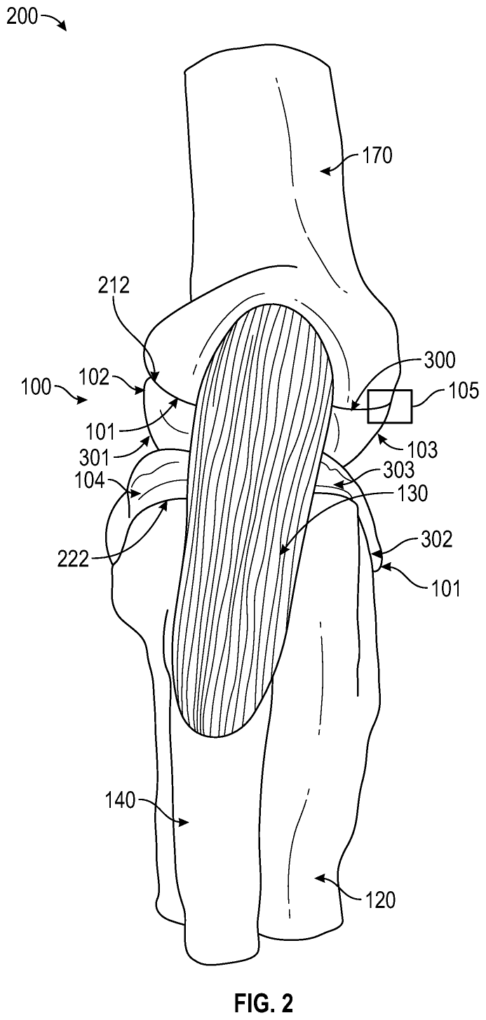

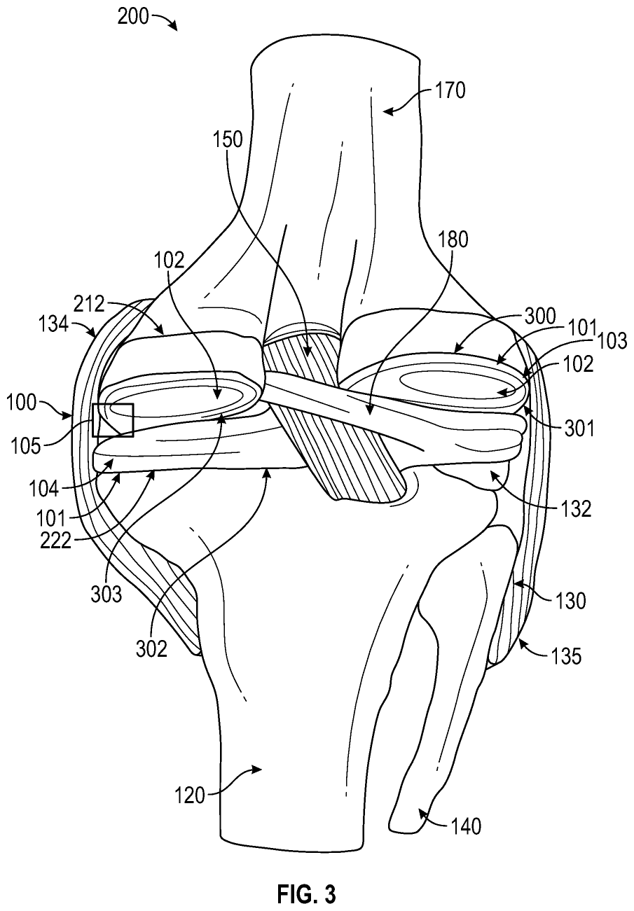

Second surface 302 of insert 104 may be secured to articular surface 222 of limb joint 200 using a biocompatible adhesive 101. Suitable biocompatible adhesives include, but are not limited to cyanoacrylates and UV curable adhesives. In aspects, the biocompatible adhesive may be a liquid, gel or spray, or in the form of a biocompatible adhesive film. Alternatively, the usage of surgical screws or other fasteners may be used to secure second surface 302 of insert 104 to the articular surface 222 of limb joint 200. First surface 303 of insert 104 receives the shock and pressure associated with the articulating limb joint, as transferred by first surface 301 of condyle 102 which is positioned on opposing surface 212 of limb joint 200. First surface 303 of insert 104 serves as a plateau upon which first surface 301 of condyle 102 may efficiently move to provide a smooth, lubricated surface for articulation and to facilitate the transmission of loads with a low frictional coefficient. Inverting the placement of condyle 102 and insert 104 such that condyle 102 is placed on articular surface 222 and insert 104 is placed on opposing surface 212 would lead to rapid degradation of the joint, the development of compromised lower lumbar segments, and, ultimately, the development of potentially chronic lower lumbar pain. After implant, first surface 303 of insert 104 and first surface 301 of condyle 102 are in a contact relationship with one another such that they may efficiently allow limb joint 200 to articulate between flexed (FIG. 1) and extended (FIGS. 2 and 3) positions as the joint is utilized during physical activity and in a resting position.

Condyle 102 may be composed of any biocompatible material capable of being formed into a shape of a naturally occurring synovial condyle and containing a fluid under the forces experienced during normal physical activity of the joint into which the present system is being employed. In aspects condyle 102 is made from a material that mimics the properties of a patient's naturally occurring synovial condyle, such as flexibility, strength and durability. Suitable materials include, but are not limited to rubberized materials, such as silicone rubber. Condyle 102 is configured to hold biocompatible fluid 103, which may be, in certain aspects, any fluid that mimics natural synovial fluid. Suitable biocompatible fluids include, but are not limited to, synthetic synovial fluids such as hyaluronan, glucosamine, and biomimetic synovial fluids. Second surface 300 of condyle 102 may be secured to opposing surface 212 of limb joint 200 through the use of a biocompatible adhesive 101 using the adhesives described above. Condyle 102 replaces and performs the function of a patient's synovial membrane which may be damaged by physical trauma or disease, or which may simply be worn out. Condyle 102 is filled with a fluid that performs the same function as the synovial fluid. Condyle 102, once filled with the fluid, serves to act as a cushion upon the end portion of bones and, ultimately, to significantly reduce the amount of friction created when a joint is articulated during movement. Condyle 102 may be periodically refilled with biocompatible fluid 103 as needed through an injection port 105 disposed along an exterior surface of first surface 301 of condyle 102. Once filled with biocompatible fluid 103, condyle 102 may possess a self-sealing quality to restore the condyle 102 to a pre-injected form. Condyle 102 is disposed upon opposing surface 212 of limb joint 200 to provide cushioning when pressure is placed upon first surface 303 of insert 104 and, ultimately, to promote efficient joint movement. The present joint replacement system 100 provides the benefit of not only delaying gradual wear and tear of the limb joint 200, but also significantly reducing the possibility and severity of lower lumbar pain development. In cases of patients who suffer from bone disease (e.g., osteoporosis), an additional abutment and/or bracing may be necessary as the limb joint 200 begins to shrink as a result of the disease thus, causing insert 104 to potentially come loose and become uncomfortable.

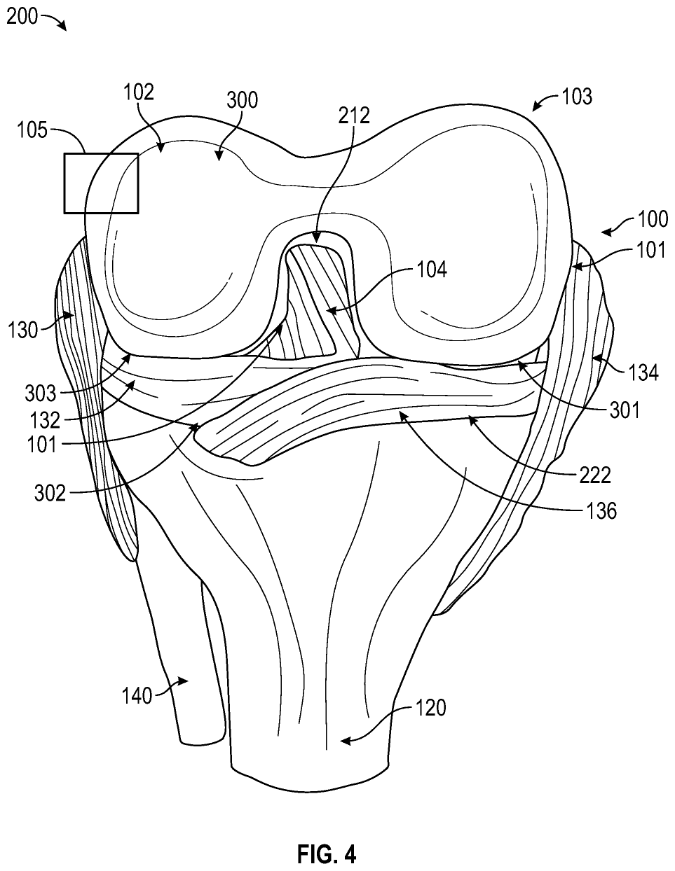

Referring to FIGS. 2 and 4, a diagram of a lateral to medial and anterior non-flexion view of the joint replacement system 100 of FIG. 1 is shown. In the extended position, first surface 301 of condyle 102 is in direct contact with first surface 303 of insert 104. The pressure placed upon first surface 301 of condyle 102 while in the extended position is distributed across first surface 303 of insert 104 such that a patient will not experience joint discomfort, the degradation of the joint will be significantly decelerated, and the likelihood of lower lumbar pain development will be minimized. The positioning of first surface 301 of condyle 102 on the surface directly opposing first surface 303 of insert 104 promotes efficient limb joint 200 articulation and, ultimately, reduces the likelihood of harmful friction occurring within the limb joint 200.

Referring to FIG. 4, a diagram of an anterior flexion view of condyle 102 filled with biocompatible fluid 103 positioned upon an articular surface 222 of limb joint 200 is shown. FIG. 4 depicts the physical relationship between first surface 301 of condyle 102 and first surface 303 of insert 104. First surface 303 of insert 104 is configured to absorb any shock or pressure that is placed upon first surface 301 of condyle 102 when a patient moves or otherwise utilizes their joint.

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate aspects, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed. Like reference numerals may refer to similar or identical elements throughout the description of the figures.

The phrases “in an aspect,” “in aspects,” “in various aspects,” “in some aspects,” or “in other aspects” may each refer to one or more of the same or different example Aspects provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The aspects described with reference to the attached drawing figures are presented only to demonstrate certain examples of the disclosure. Other elements, steps, methods, and techniques that are insubstantially different from those described above and/or in the appended claims are also intended to be within the scope of the disclosure.

1. A joint replacement system comprising:
an insert configured to be disposed on an articular surface of a limb joint;
a biocompatible fluid, wherein the biocompatible fluid includes hyaluronic acid; and
a condyle configured to contain the biocompatible fluid and to be disposed on an opposing surface of the limb joint,
wherein the condyle and the insert are aligned to enable articulation of the limb joint, and
wherein a first surface of the insert is continuous and configured to contact the condyle and a second opposing surface of the insert is configured to be disposed on the articular surface of the limb joint.

2. The joint replacement system of claim 1, wherein the insert is secured on the articular surface of the limb joint by a biocompatible adhesive.

3. The joint replacement system of claim 2, wherein the biocompatible adhesive is a biocompatible adhesive film.

4. The joint replacement system of claim 1, wherein the insert is made at least in part from polytetrafluoroethylene.

5. The joint replacement system of claim 1, wherein the limb joint is a knee joint.

6. The joint replacement system of claim 1, wherein the condyle is configured to selectively receive an amount of the biocompatible fluid therein.

7. The joint replacement system of claim 1, wherein the biocompatible fluid includes a synthetic synovial fluid.

8. The joint replacement system of claim 1, wherein the condyle is made at least in part from a rubberized biocompatible material.

9. The joint replacement system of claim 1, wherein the insert and the condyle are configured to articulate against one another.

10. The joint replacement system of claim 9, wherein the insert and the condyle are configured to be in a contact relationship with one another when the limb joint is in both a flexed and extended position.

11. A condyle configured for being disposed on an opposing surface of a limb joint, the condyle comprising:
an exterior surface defining a recess configured to separate a first curvilinear portion of the condyle from a second curvilinear portion of the condyle, the recess disposed between the first and second curvilinear portions of the condyle;
an interior surface defining a cavity for containing a biocompatible fluid;
a connector member disposed within the cavity between the first and second curvilinear portions of the condyle, the connector member configured to enable fluid communication within the cavity between the first and second curvilinear portions of the condyle; and
an injection port disposed on the exterior surface of the condyle.

12. The condyle of claim 11, wherein the exterior surface of the condyle is made at least in part of a rubberized biocompatible material.

13. The condyle of claim 11, wherein the biocompatible fluid is a synthetic synovial fluid.

14.-17. (canceled)

18. The joint replacement system of claim 1, wherein during articulation of the limb joint, a surface of the condyle is configured to distribute a pressure applied thereon across a surface of the insert, the surface of the insert configured to absorb the applied pressure.

19. A joint replacement system comprising:
an insert configured to be disposed on an articular surface of a limb joint;
a biocompatible fluid, wherein the biocompatible fluid includes hyaluronic acid; and
a condyle configured to contain the biocompatible fluid and to be disposed on an opposing surface of the limb joint, the condyle including:
an exterior surface defining a recess configured to separate a first curvilinear portion of the condyle from a second curvilinear portion of the condyle, the recess disposed between the first and second curvilinear portions of the condyle;
an interior surface defining a cavity for containing a biocompatible fluid; and
a connector member disposed within the cavity between the first and second curvilinear portions of the condyle, the connector member configured to enable fluid communication within the cavity between the first and second curvilinear portions of the condyle,
wherein the condyle and the insert are aligned to enable articulation of the limb joint.

20. The joint replacement system of claim 19, wherein a first surface of the insert is continuous and configured to contact the condyle and a second opposing surface of the insert is configured to be disposed on the articular surface of the limb joint.

21. The joint replacement system of claim 19, wherein during articulation of the limb joint, a surface of the condyle is configured to distribute a pressure applied thereon across a surface of the insert, the surface of the insert configured to absorb the applied pressure.

22. The joint replacement system of claim 1, wherein the limb joint is a knee joint.
